(19) United States
(12) Patent Application Publication
(10) Pub. No.: US 2019/0353739 A1
(43) Pub. Date: Nov. 21, 2019

(54) METHOD AND APPARATUS FOR RECONSTRUCTING MAGNETIC RESONANCE TOMOGRAPHY IMAGES WITH VARIABLE TIME RESOLUTION

(71) Applicant: Siemens Healthcare GmbH, Erlangen (DE)

(72) Inventors: Wilhelm Horger, Schwaig (DE); Ralf Kartaeusch, Erlangen (DE); Dominik Paul, Bubenreuth (DE)

(73) Assignee: Siemens Healthcare GmbH, Erlangen (DE)

(21) Appl. No.: 16/412,851

(22) Filed: May 15, 2019

(30) Foreign Application Priority Data
May 15, 2018 (EP) 18172265

(51) Int. Cl. G01R 33/561; G16H 30/40

(52) U.S. Cl. CPC G01R 33/5611; G01R 33/4818; G16H 30/40

(57) ABSTRACT

In a magnetic resonance (MR) method and apparatus for reconstructing MR tomography images with variable time resolution, a preliminary reconstruction of preview images takes place, using raw data acquired in a closed time period at different times from the same body region of a patient. Scan data are acquired from the preview images and/or from the raw data, the scan data quantitatively representing the introduction of a contrast medium over a time span. Diagnostic reconstruction parameters are then specified, which designate time portions in which reconstruction is to be conducted with different time resolutions, based on the scan data. The reconstruction of diagnostic MR tomography images then takes place, using the diagnostic reconstruction parameters.

### BACKGROUND OF THE INVENTION

### Field of the Invention

The invention relates to a method for reconstructing magnetic resonance tomography imaging with a variable time resolution, as well as a reconstruction computer, an apparatus control computer and a corresponding diagnostic station, and a medical imaging apparatus that implement such a method.

### Description of the Prior Art

Imaging dynamic processes, such as those involving the introduction of a contrast medium, by magnetic resonance (MR) tomography presents challenges due to the relatively slow recording (data acquisition) times of the MR scanner. One possibility for addressing those challenges is the use of Compressed Sensing GRASP-VIBE.

Compressed Sensing GRASP-VIBE is a technique that uses a radial scan with a golden-angle distribution. This technique is typically used to image dynamic processes such as the introduction of contrast media. The acquisition scheme enables images to be reconstructed at arbitrary time points with an arbitrary time resolution. The abbreviation GRASP stands for “Golden-angle RAdial Sparse Parallel” and VIBE stands for “Volumetric Interpolated Breath-hold/Brain Examination”. The recording method corresponds to a VIBE sequence with a radial trajectory in which a Cartesian encoding is carried out in the slice direction. K-space radii are recorded continuously with the spacing of the golden-angle (see also “Golden-angle radial sparse parallel MRI: Combination of Compressed Sensing, parallel imaging, and golden-angle radial sampling for fast and flexible dynamic volumetric MRI”, L. Feng et al., Magn Reson Med. 2014 September; 72(3):707-17).

The GRASP-VIBE method can be used to acquire MR data that represent the introduction of a contrast medium. The dynamics of the contrast medium introduction vary significantly over the duration of the scan. Therefore, the scan is typically subdivided into different phases, in which the temporal resolution of the expected dynamics must be individually adapted. For example, in the arterial phase a very high time resolution should be used, and in the “late phase” in which hardly any changes take place, only a very low time resolution is needed.

The time resolution has a direct effect on the quality of the reconstructed images. If a relatively low time resolution is selected, the image becomes less sharp. If a relatively high time resolution is selected, the image becomes sharper, but the noise, and streaking artifacts that are typical of radial scans due to the more severe undersampling, become increased. Furthermore, the time resolution should not be selected constantly high over the scan, since otherwise very many images would be generated, resulting in a correspondingly long assessment.

It is problematic that the time points of the contrast medium introduction significantly vary according to lesion, vessel or organ. Therefore, the start and end points of the phases of different time resolution can only be roughly estimated.

In order to define phases with different time resolutions, the conventional procedure is, before the reconstruction, that either a time point for the introduction is estimated manually, or an automatic detection of the introduction of the contrast medium in the aorta is carried out. In both cases a high time resolution is specified at the corresponding time points. With this conventional approach, the resulting time points are often not the optimum time point for some of the lesions or vessels, which represents a great disadvantage.

### SUMMARY OF THE INVENTION

An object of the present invention is to overcome the disadvantages of the prior art and to make available a method and a reconstruction apparatus for improved reconstruction of magnetic resonance tomography images with a variable time resolution. The present invention also encompasses a control computer and a diagnostic station and a medical imaging apparatus that are designed to implement the method according to the invention.

The inventive method for reconstructing magnetic resonance tomography images with variable time resolution includes the following steps.

A computer creates or is provided with raw data. In this step, a new data set of magnetic resonance tomography raw data can be created by the computer operating an MR scanner so as to acquire the raw data. It is also possible, depending upon the application case, to provide the computer with an existing data set, for example, by downloading a corresponding file from a data store and/or transferring the file from the data store via a network. This data set is composed of a series of raw data that were acquired by operation of a magnetic resonance tomography scanner, at different times within a closed time period, from the same body region of a patient. The magnetic resonance tomography raw data are or were generated by the use of an MR data acquisition sequence that enables a reconstruction with a sliding window, that is, a variable time resolution. This can be, for example, a GRASP recording method, in particular a GRASP-VIBE recording method.

A preliminary reconstruction of preview images then takes place. In this step, preview images are reconstructed from the magnetic resonance tomography raw data. A reconstruction algorithm known from the prior art can be used. With regard to the introduction of a contrast medium, for example, known pre-settings for different time points can be used.

Scan data are then acquired. In this step, scan data that represent quantitatively the introduction of a contrast medium over a timespan are acquired from the preview images and/or from the raw data (preferably from the raw data). The scan data are, for example, data from a GRASP scan, e.g. the variation of the energy or the signal intensity in the k-space center over time. The presence of a contrast medium provides for a faster relaxation of the excited nuclear spins, which is expressed in an increased energy. Preferably, for this purpose, a region is selected in the preview image, e.g. by an imaged crosshair or a “pixel lens”. From the scan data, therefore, what contrast medium concentration is present in a region at which time point can be derived.

The scan data can be represented and displayed in the form of a contrast medium curve. In the case of a contrast medium curve, according to the prior art, the signal intensity in the k-space center is represented filtered over time. Preferably, therein the contrast medium curve is determined as a graphical representation of the scan data over time and is preferably displayed live (in real time). If a region has been selected (e.g. by use of a selection tool such as a “pixel lens” in a preview image), a second (comparison) contrast medium curve can also be generated on the basis of a second region (comparison area) and both contrast medium curves can be displayed.

Main reconstruction parameters are then specified. These main reconstruction parameters serve for a reconstruction of the recorded raw data and include time portions in which reconstruction takes place with different time resolutions. They differ from the parameters used during the preliminary reconstruction by being dependent on (formulated using) information that has been obtained from the scan data. The main reconstruction parameters therefore represent improved parameters in relation to the parameters used in the preliminary reconstruction. According to the above example, the main reconstruction parameters preferably result on the basis of a contrast medium curve which has been created from the scan data.

According to a preferred embodiment, the main reconstruction parameters are stipulated manually by a user. The possibility is thereby provided to the operator to select time portions on the basis of the scan data. This allows the operator to select, for example, those time portions in the scan data in which a high level of dynamics prevails. Preferably, via a corresponding user interface, the operator selects the time point using a contrast medium curve. This selection can also be made automatically, for example, on the basis of the shape of a contrast medium curve.

In place of a time point that is reconstructed with a time resolution, a time span can be selected in which a number of “time points” are reconstructed with the same time resolution. A “time point” corresponds to a 3D volume or a stack of slices with images for one time point.

The main reconstruction takes place. In this step, magnetic resonance tomography recordings, that is, images that are intelligible to a diagnostician, are reconstructed from the magnetic resonance tomography raw data. The reconstruction is implemented using the preceding specified main reconstruction parameters, that is, the time portions available therefrom.

In the main reconstruction, therefore, compared with the prior art, a reconstruction is started retrospectively with new parameters (the main reconstruction parameters). It should be noted that a reconstruction on the currently available systems with a reconstruction time of up to 5 min takes a very long time. It is therefore preferred for the reconstruction to be restricted to the slices of interest. If a lesion extends, for example, to only through a few slices, or if one slice is sufficient to classify it, the reconstruction can be carried out in a few seconds.

In summary, the invention uses information taken from preview images for the main reconstruction of the diagnostic images. The “main reconstruction” thus can be considered a “diagnostic reconstruction.”

An inventive reconstruction computer for reconstructing magnetic resonance tomography images with variable time resolution includes the following components.

A data interface acquires a data set composed of a series of magnetic resonance tomography raw data recorded in a closed time period at different times from the same body region of a patient (see above).

A preliminary reconstruction unit is configured to implement a preliminary reconstruction of preview images as described above from the magnetic resonance tomography raw data.

An acquisition unit acquires scan data from the preview images and/or from the raw data, which quantitatively represent the introduction of a contrast medium over a timespan. This acquisition unit is preferably configured to generate a contrast medium curve and particularly preferably also to provide it (at least the data relating thereto), as an output in graphical form.

A parameter specifies main reconstruction parameters having time portions in which reconstruction is carried out with different time resolutions on the basis of the scan values. This parameter unit can be linked to an input unit that receives a manual selection by a diagnostician. It is also possible for the parameter unit to automatically determine the time portions from regions of the scan data with high dynamics in which different time resolutions are to be used for a reconstruction.

A main reconstruction unit is configured to execute the main reconstruction of magnetic resonance tomography images from the magnetic resonance tomography raw data on the basis of the main reconstruction parameters. This main reconstruction unit can be the same unit that forms the preliminary reconstruction unit, so that, the same hardware or software modules can be used. In the main reconstruction, however, during the reconstruction, the main reconstruction parameters must be accessed.

Given suitably fast reconstruction hardware and a reduction to one slice or a few slices (see above), a corresponding user interface for manual specification of the main reconstruction parameters can preferably also displays “live” images (possibly with reduced inplane resolution) of the respective current time point, in order to enable the user to make an optimum selection of a desired time point. This procedure also provides new possibilities in the original preliminary reconstruction. Since the images with the high dynamic are only retrospectively generated live, the original preliminary reconstruction can be undertaken with significantly lower time resolution, if desired. As a result, this becomes faster and the number of images to be assessed is reduced.

An advantage of the invention is that an optimum contrast can be created for lesions, vessels or organs. The inventive method also enables images, which have only a limited diagnostic valve due to artifacts, to be optimized after they have been produced. In an exemplary embodiment in which a very high time resolution has been selected for the arterial phase, and due to the physiology and/or movement of the patient, streaking artifacts have been induced, the time resolution can be subsequently reduced, enabling an improved diagnosis. If, for example, the patient moves during the scan, this is expressed as artifacts in the images. If the time points are selected so that the data that were acquired during the movement are not used, the artifacts can be removed.

An inventive control computer for controlling a magnetic resonance tomography system has an inventive reconstruction computer and/or is itself configured for carrying out an inventive method.

An inventive diagnostic station (for example, a powerful computer console) is or can be coupled to a magnetic resonance tomography system. It includes an inventive reconstruction computer and/or is itself configured for carrying out an inventive method.

An inventive medical imaging system (apparatus) has an inventive reconstruction computer and a magnetic resonance tomography scanner, whereby the reconstruction computer is preferably present in the medical imaging apparatus in the form of an inventive diagnostic station and/or an inventive control computer.

A majority of the aforementioned components of the reconstruction computer, the diagnostic station and/or the control computer can be realized entirely or partially in the form of software modules in a processor of the reconstruction computer, diagnostic station, or control computer. A realization largely through software has the advantage that conventionally used control computer can easily be upgraded with a software update in order to operate according to the invention.

Therefore, the present invention also encompasses a non-transitory, computer-readable data storage medium encoded with programming instructions (program code) that, when executed in one or more of the computers of the type described above, cause the method according to the invention, as also described above, to be executed, when the storage medium is loaded into one of those computers, or is loaded in a distributed manner into multiple computers.

Such a storage medium can also have, in addition to the code program, for executing the inventive method, further codes, such as documentation code, and/or additional components including hardware components such as hardware keys (dongles, etc.) in order to use the program code.

The computer-readable medium can be a memory stick, a hard disk or another transportable or firmly installed data carrier.

Preferably a (large) number of intermediate images are reconstructed, in particular in the context of a preliminary reconstruction of preview images. These intermediate images should have the highest possible time resolution (preferably the maximum possible time resolution) wherein artifacts occurring thereby are initially incidental. In the main reconstruction, these already existing intermediate images are then used. The magnetic resonance tomography images are reconstructed by averaging the intermediate images according to the main reconstruction parameters. Therefore, initially, very many images are generated, their number being reduced again in the main reconstruction through the averaging to the desired time resolution.

Preferably, a display of the scan values takes place, particularly preferably in the form of a contrast medium curve, together with preview images. A preferred reconstruction apparatus has a display unit for displaying the scan data, preferably the contrast medium curve, in particular together with preview images.

Preferably, a selection of time portions, and in particular also a selection of the time resolution within the time portions, is carried out by a user, preferably on the basis of a displayed contrast medium curve, in particular together with the displayed preview images.

For the definition of a time portion, a time point (such as a previously defined length of a time portion) is preferably included and preferably additionally a time span (in the case of a time portion having a variable length) in the main reconstruction parameters. It is therefore possible, where relevant, in addition to the actual time point, also to select a time span. This provides further advantage that the degree of undersampling or image noise can be influenced and optimized.

Preferably, a time portion is pre-selected by the use of a bolus detection.

Preferably, the raw data are recorded with a GRASP recording sequence (in particular radial VIBE), preferably with a Compressed Sensing GRASP-VIBE recording sequence.

Preferably, the main reconstruction parameters additionally contain information relating to (for an investigation) relevant slices which are to be reconstructed at a particular time resolution in a particular time portion. On the basis of these main reconstruction parameters, a main reconstruction of the relevant slices is then carried out.

Preferably, the reconstruction computer comprises an output controller configured to control the display unit such that a representation of graphical elements takes place, which represent a selection of time portions and, in particular, also the time resolution within the time portions by a user. The output controller is also configured to cause display of further preview images that represent the preliminary reconstruction of magnetic resonance tomography images.

### BRIEF DESCRIPTION OF THE DRAWINGS

FIG. 1 schematically illustrates a magnetic resonance tomography apparatus according to an exemplary embodiment of the invention.

FIG. 2 is a schematic flowchart of the basic steps of the inventive method.

FIG. 3 shows an example of a preview image in accordance with the invention.

FIG. 4 shows an example of a contrast medium curve that is used in accordance with the invention.

### DESCRIPTION OF THE PREFERRED EMBODIMENTS

In the following figures only elements that are essential to the invention or are helpful for its comprehension are shown.

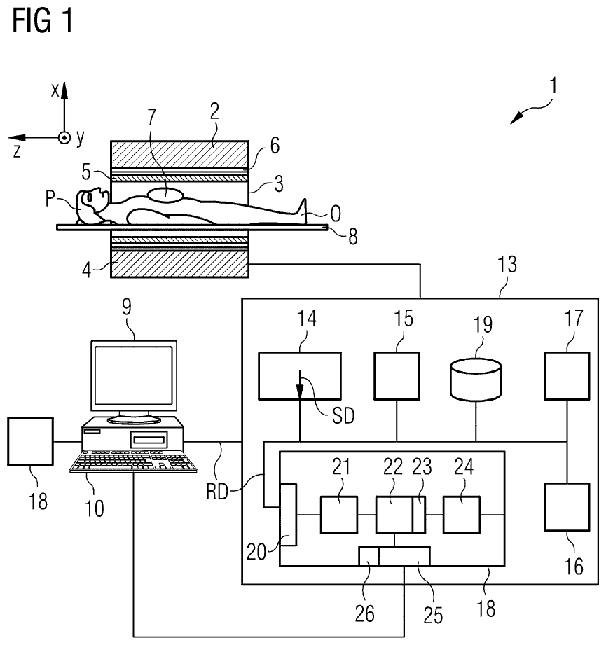

FIG. 1 schematically shows a magnetic resonance tomography apparatus 1, which includes the actual magnetic resonance scanner 2 with an examination volume 3 or patient tunnel in which a patient P or test subject is positioned on a table 8. The actual examination object O is situated in the patient P.

The magnetic resonance scanner 2 is typically equipped with a basic field magnet 4, a gradient system 6 and an RF transmitting antenna 5 and an RF receiving antenna 7. In the exemplary embodiment shown, the RF transmitting antenna 5 is a whole body coil installed in the magnetic resonance scanner 2, whereas the RF receiving antenna 7 is formed by local coils arranged on the patient P or test subject (symbolized in FIG. 1 by a single local coil). Fundamentally, however, the whole body coil can also be used as an RF receiving antenna system and the local coils can be used as the RF transmitting antenna system, provided these coils are each switchable into different operating modes. The basic field magnet 4 is typically configured herein so that it generates a main magnetic field in the longitudinal direction of the patient, i.e. along the longitudinal axis of the magnetic resonance scanner 2, extending in the z-direction. The gradient coil system 6 typically has individually controllable gradient coils in order to be able to switch (activate) gradients in the x, y or z directions independently of each other. In addition, the magnetic resonance scanner 2 has shim coils (not shown), which can be configured in the usual manner.

The magnetic resonance tomography scanner 2 shown in FIG. 1 is a whole body system with a patient tunnel into which a patient P can be completely introduced. In principle, however, the invention can also be used with other magnetic resonance tomography systems, e.g. with laterally open, C-shaped housings. It is only essential that suitable recordings of the examination object O can be prepared. The magnetic resonance tomography apparatus 1 further has a central control computer 13 that controls the MR apparatus 1. This central control computer 13 comprises a sequence controller 14 that controls the sequence of radio-frequency (RF) pulses and gradient pulses according to a selected pulse sequence PS within a scan session, so that, for example, a GRASP-VIBE recording method is executed. Typically, different control protocols are stored as pulse sequences PS for different scans or scan sessions in a memory 19 and can be selected by an operator (and if needed, possibly changed), and then used for carrying out the scan.

For producing the individual RF pulses of a pulse sequence PS, the central control computer 13 has a radio-frequency transmitting device 15, which generates the RF pulses, and amplifies and feeds them via a suitable interface (not shown in detail) into the RF transmitting antenna 5. For the control of the gradient coils of the gradient coil system 6 in order to switch the gradient pulses according to the pre-defined pulse sequence PS, the control computer 13 has a gradient system interface 16. The sequence controller 14 communicates in a suitable manner, for example, by transmitting sequence control data SD with the radio-frequency transmitting device 15 and the gradient system interface 16 for carrying out the pulse sequence PS.

The control computer 13 also has a radio-frequency receiving device 17 (also communicating in a suitable manner with the sequence controller 14), in order to receive magnetic resonance signals within the readout window pre-determined by the pulse sequence PS in a coordinated manner, detected by the RF receiving antenna 7, so as to acquire the raw data.

The basic manner of radiation of RF pulses and the switching of gradient pulses, in order to acquire raw and the reconstruction of MR images or parameter maps therefrom, is known to those skilled in the art and therefore need not be described in more detail herein.

A reconstruction apparatus 18 accepts the acquired raw data RD and reconstructs therefrom magnetic resonance recordings B in the context of the invention. This image data can be stored, for example, in the memory 19.

The reconstruction computer 18 has a data interface 20 for acquiring a data set with a series of magnetic resonance tomography raw data RD acquired in a closed time period at different times from the same body region of the patient P. This data interface 20 is in data communication with a preliminary reconstruction computer 21, which is configured to undertake a preliminary reconstruction of the magnetic resonance tomography raw data RD so as to produce preview images VB.

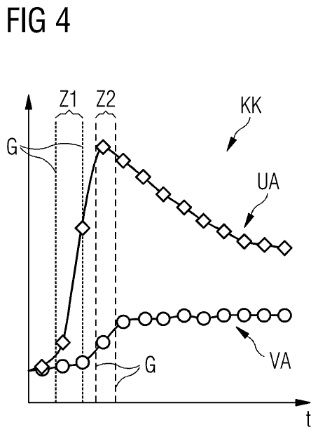

An acquisition unit 22 acquires scan data MD from the preview images VB and/or from the raw data RD. The scan data MD quantitatively represent the introduction of a contrast medium over a timespan. The scan data can be, for example, in the form of a contrast medium curve KK, as shown in FIG. 4. In this example, the contrast medium curve KK is shown together with a preview image VB of a display unit 25. This display unit 25 can be, as shown in this example, an interface that creates a data connection with a terminal 10. It can also be an independent display.

The reconstruction computer 18 has, in this example, an output control unit 26 for controlling the display unit 25. The output control unit 26 enables a representation of graphical elements, which represent a selection of time portions by a user. It can also be configured to display further preview images VB that represent a preliminary reconstruction of magnetic resonance tomography images B.

A parameter unit 23 permits a specification of main reconstruction parameters HP, which designate time portions in which reconstruction is carried out with different time resolutions on the basis of the scan values. On the basis of these main reconstruction parameters HP, a main reconstruction computer 24 carries out a main reconstruction of the magnetic resonance tomography images B from the magnetic resonance tomography raw data RD.

Operation of the central control computer 13 can take place via a terminal 10 with a display unit 9, via which therefore the entire magnetic resonance tomography apparatus 1 can be operated by an operating person. On the display unit 9, the magnetic resonance tomography recordings B can also be displayed, and via the terminal 10, scans can be planned and started and protocols P can be selected and, if required, modified. In this case, the terminal 10 is configured as a diagnostic station 10 and is provided with an inventive reconstruction computer 18 designed and operating as described above.

The inventive magnetic resonance tomography apparatus 1 and the control computer 13 can also have further components that are not shown in detail herein, but are typically present on such systems. These include, for example, a network interface in order to connect the overall system to a network (and e.g. further diagnostic stations) and to be able to exchange raw data and/or image data or parameter maps, as well as further data such as patient-relevant data or control protocols.

The radiation of RF pulses and the creation of gradient fields in order to acquire raw data, and the reconstruction of magnetic resonance tomography images are known to those skilled in the art, and therefore need not be described in more detail herein. Similarly, a variety of scan sequences, for example, EPI scan sequences or other scan sequences for generating diffusion-weighted images, are known in principle to those skilled in the art.

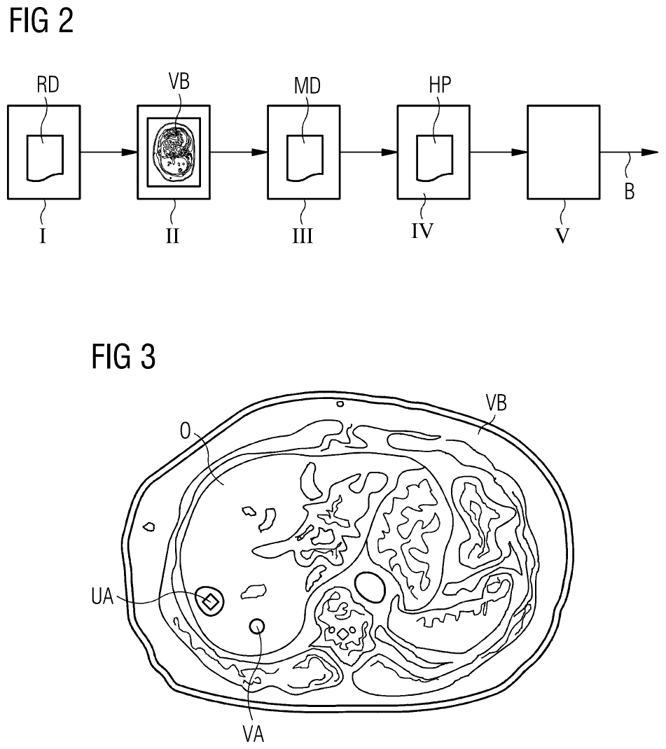

FIG. 2 shows, in the form of a block diagram, a schematic representation of the inventive method for reconstructing magnetic resonance tomography images with variable time resolution.

In step I, the creation or preparation of a data set comprising a series of magnetic resonance tomography raw data RD which has been recorded in a closed time period at different times from the same body region of a patient P, e.g. by means of a GRASP-VIBE recording method, takes place.

In step II, a preliminary reconstruction of preview images VB from the magnetic resonance tomography raw data RD takes place.

In step III, an acquisition of scan data MD from the preview images VB and/or from the raw data RD which quantitatively represents the introduction of a contrast medium over a timespan takes place. From this scan data, a contrast medium curve KK can be created which can be displayed.

In step IV, on the basis of the scan data MD, a specification of main reconstruction parameters HP comprising time portions in which reconstruction is carried out with different time resolutions takes place.

The main reconstruction parameters HP can be, for example, specified manually. For this purpose, for example, a display of the scan data MD in the form of a contrast medium curve KK together with a preview image VB takes place. Such a display is shown, for example, in FIGS. 3 and 4.

A diagnostician can specify, for example, via a graphical interface on which movable selection instruments are represented, the main reconstruction parameters HP.

In step V, there follows a main reconstruction of magnetic resonance tomography recordings B from the magnetic resonance tomography raw data RD, on the basis of the main reconstruction parameters HP.

FIG. 3 shows an example of a preview image. Shown here is a sectional image through a human liver, which represents the examination object O. In this liver, a region of a lesion is drawn in (encircled) in which an examination area UA is drawn. In this examination area UA, it is of interest when a contrast medium is introduced. For comparison, data of a comparison area VA is also shown.

FIG. 4 shows an example of a contrast medium curve KK. In a coordinate system in which the X-axis corresponds to the time axis t, the measured contrast medium quantity in the examination area UA over time and for comparison, also that of the comparison area are each plotted on the Y-axis. The variation in the lesion (that is, the examination area UA) deviates from that in the healthy tissue. A diagnostician can now delimit, by movable limit indicators G, time portions Z1, Z2, within which a pre-determined time resolution is to be used in the reconstruction. In this example, there exist two time portions Z1, Z2 which are marked by different limit indicators G (different line dashing). In a first time portion Z1, reconstruction is to be carried out with a greater time resolution than in the second time portion Z2. A diagnostician can, for example, displace the limit indicators G on the time axis t and so specifically select those time portions Z1, Z2 in the contrast medium curve KK in which reconstruction is to take place with the desired time resolution.

It should again be noted that the use of the indefinite article “a” or “an” does not preclude the features in question from being present plurally. Similarly, the expressions “unit” and “module” do not preclude the components in question from consisting of a plurality of cooperating partial components with can also be spatially distributed.

Although modifications and changes may be suggested by those skilled in the art, it is the intention of the Applicant to embody within the patent warranted hereon all changes and modifications as reasonably and properly come within the scope of the Applicant's contribution to the art.

1. A method for reconstructing magnetic resonance (MR) tomography images with variable time resolution, comprising:
providing a computer system with a data set comprising a series of MR tomography raw data acquired in a closed time period at different times from a same body region of a patient;
in said computer system, implementing a preliminary construction of preview images from the MR tomography raw data;
in said computer system, acquiring scan data from at least one of said preview images and said raw data, said scan data quantitatively representing an introduction of a contrast medium into the patient over a time span;
with said computer system, receiving or producing diagnostic reconstruction parameters comprising time portions in which reconstruction of MR image data are to be implemented with respective different time resolutions, based on said scan data; and
in said computer system, using said diagnostic reconstruction parameters to implement a diagnostic reconstruction of said MR tomography raw data into diagnostic MR tomography images, comprising said MR image data.

2. A method as claimed in claim 1 comprising implementing said preliminary reconstruction in order to initially reconstruct intermediate images with a maximum possible time resolution and, in said diagnostic reconstruction, reconstructing said MR tomography images by averaging said intermediate images according to said diagnostic reconstruction parameters.

3. A method as claimed in claim 1 comprising displaying said scan data at a display in communication with said computer system.

4. A method as claimed in claim 3 comprising displaying said scan data as a contrast medium curve representing progress of said contrast medium in said patient, together with said preview images.

5. A method as claimed in claim 1 comprising selecting said time portions by a manual entry into said computer system.

6. A method as claimed in claim 1 comprising defining said time portion as a beginning time point and a subsequent time span designated in said diagnostic reconstruction parameters.

7. A method as claimed in claim 1 comprising providing said computer system with said data set that was acquired by executing a GRASP imaging sequence.

8. A method as claimed in claim 7 wherein said GRASP imaging sequence is a Compressed Sensing GRASP-VIBE imaging sequence.

9. A method as claimed in claim 1 wherein said diagnostic reconstruction parameters additionally comprise information defining relevant slices of the patient that are to be reconstructed with a predetermined time resolution in a predetermined time portion, and implementing said diagnostic reconstruction to reconstruct said MR tomography images of said slices.

10. A magnetic resonance (MR) tomography apparatus comprising:
an MR data acquisition scanner;
a computer system configured to operate said MR data acquisition scanner to acquire a data set comprising a series of MR tomography raw data in a closed time period at different times from a same body region of a patient;
said computer system being configured to implement a preliminary construction of preview images from the MR tomography raw data;
said computer system being configured to acquire scan data from at least one of said preview images and said raw data, said scan data quantitatively representing an introduction of a contrast medium into the patient over a time span;
said computer system being configured to receive or produce diagnostic reconstruction parameters comprising time portions in which reconstruction of MR image data are to be implemented with respective different time resolutions, based on said scan data; and
said computer system being configured to use said diagnostic reconstruction parameters to implement a diagnostic reconstruction of said MR tomography raw data into diagnostic MR tomography images, comprising said MR image data.

11. A non-transitory, computer-readable data storage medium encoded with programming instructions, said storage medium being loaded into a computer system and said programming instructions causing said computer system to:
receive a computer system with a data set comprising a series of MR tomography raw data acquired in a closed time period at different times from a same body region of a patient;
implement a preliminary construction of preview images from the MR tomography raw data;
acquire scan data from at least one of said preview images and said raw data, said scan data quantitatively representing an introduction of a contrast medium into the patient over a time span;
receive or produce diagnostic reconstruction parameters comprising time portions in which reconstruction of MR image data are to be implemented with respective different time resolutions, based on said scan data; and
use said diagnostic reconstruction parameters to implement a diagnostic reconstruction of said MR tomography raw data into diagnostic MR tomography images, comprising said MR image data.
